(19) United States
(12) Patent Application Publication
(10) Pub. No.: US 2001/0019713 A1
(43) Pub. Date: Sep. 6, 2001

(54) Novel interleukin compositions and methods of use

(76) Inventors: Beverly A. Blazar, Providence, RI (US); Andrew C. Webb, Milton, MA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/760,541

(22) Filed: Jan. 16, 2001

Related U.S. Application Data
(63) Continuation of application No. 08/287,387, filed on Aug. 8, 1994, now abandoned.
Application No. 08/287,387 is a continuation-in-part of application No. 08/026,132, filed on Feb. 11, 1993, now abandoned.

(51) Int. Cl. A61K 39/395; C07K 1/00; C07K 14/00; C07K 17/00

(52) U.S. Cl. 424/130.1; 530/351

(57) ABSTRACT

A novel immunoregulatory factor, designated IL-X, is described which has been isolated from an Epstein-Barr virus (EBV) infected lymphoblastoid cell line. IL-X is a growth factor for EBV transformed human B lymphocytes and for murine helper T lymphocytes. Also taught are methods of raising antibodies to IL-X, and cloning of IL-X.

### CROSS-REFERENCE TO A RELATED APPLICATION

This application is a continuation-in-part of co-pending application Ser. No. 08/026,132, filed Feb. 11, 1993.

### BACKGROUND OF THE INVENTION

Epstein-Barr Virus (EBV) is a lymphotropic virus in humans that is closely associated with two malignancies, Burkitt's lymphoma, and nasopharyngeal carcinoma, as well as a lymphoproliferative disorder, infectious mononucleosis. Also, in recent years, several EBV-associated proliferative syndromes and malignancies have been described in the profoundly immunocompromised host.

There are two main types of EBV carrying B lymphocyte lines, i.e., Burkitt's Lymphoma derived (BL) and lymphoblastoid (normal lymphocyte) derived cell line (LCL). BL lines, which are derived from malignant cells in tumor biopsies, are monoclonal, usually aneuploid with a specific chromosomal translocation, bear a characteristic glycoprotein pattern, and are tumorigenic in nude mice. The LCL lines are derived from normal B cells, are polyclonal, have a normal diploid karyotype, a glycoprotein pattern similar to stimulated normal B cells, and do not grow when explanted subcutaneously into nude mice. LCLs, however, do grow in nude mice when inoculated intracerebrally, suggesting that immunological restriction is important in controlling outgrowth of EBV-carrying cells, even in a xenogeneic host. Reports of polyclonal outgrowths of karyotypically normal EBV-carrying cells in immunodeficient individuals confirm this observation (Houweling, A., P. J. Eisen, A. J. Eb [1980] Virology 105:537; Rassoulzadegan, M., M. Binetriy, F. Cuzin [1982] Nature 300:713; Treisman, R., V. Novak, J. Favoloro, R. Kamen [1981] Nature 292:595; Giovanella, B., K. Nilsson, L. Zech, O. Yim, G. Klein, J. S. Stehlin [1979] Int. J. Cancer 24:103).

In general, tumor cells develop from normal cells by a multistage process. Two critical stages include (a) immortalization, i.e., the ability to divide perpetually without exogenously supplied mitogenic stimuli, and (b) acquisition of resistance to negative homeostatic signals that normally regulate growth. These stages may be associated with cytokines because regulation of proliferation and differentiation in most eukaryotic cells is accomplished by the interaction of specific cytokines with cell surface receptors. Receptor activation is followed by transmembrane signal transduction which leads to the generation of specific second messenger molecules. These receptor dependent events result in a defined series of cytoplasmic and/or nuclear changes leading to regulation of cellular activity.

Autonomous growth, as a result of transformation associated events, occurs in normal B cells transformed in vitro by EBV, and also in B cells derived from EBV positive and negative malignancies. EBV-transformed normal B lymphocytes divide continuously in culture without help from T cells or macrophages. Factor dependent autostimulatory growth for EBV-carrying B lymphocytes has now been reported by many groups. This secreted growth enhancing activity is specific for mature lymphoid cells. In addition, immortalized EBV-carrying B cells respond differently than normal B cells to certain cytokines, e.g., they proliferate in response to TGFβ and IL-6. After EBV infection, B lymphocytes have an altered morphological appearance, produce immunoglobulin, and become independent of exogenous differentiation factors and resistant to saturation conditions in cell culture.

How lymphoid cells communicate with each other to affect cell growth, differentiation, and functional activities has been a major focus of investigation. The immune response to foreign antigens is dependent on the interactions of several different cell types, including macrophages, T, and B lymphocytes. The first described soluble growth factor of lymphoid origin, T cell growth factor (IL-2), was found in supernatants of lectin stimulated peripheral blood lymphocytes. Since the discovery of IL-2, various studies have described many additional growth factors and have begun to delineate the mechanisms controlling lymphocyte proliferation.

For the B lymphocyte, our understanding of the regulation of growth and differentiation has increased in complexity in the past few years. A plethora of factors, including BCGF (12 and 60 kD), IFNγ, TNFα, lymphotoxin, TGFβ, IL-1α, IL-1β, IL-2, IL-4, IL-5, IL-6, IL-7, IL-10, and C3 fragments have been reported to modulate the growth of human and/or murine B lymphocytes in cell culture studies. These effects include growth augmentation (BCGF), differentiation (IL-6), and inhibition of proliferation (TGFβ). It appears that many of the T lymphocyte derived immunomodulatory molecules which direct B lymphocyte activities also regulate pleomorphic T cell functions. Some affect growth (IL-2), whereas others can also cause changes in cellular differentiation (IL-3). IL-2 functions as a direct growth factor for T lymphocytes, but other factors (IL-1) are described as comitogens or “helper” factors for cells stimulated by antigen or mitogen. IL-1, however, has recently been reported to function as a direct growth factor for one T cell sub-clone (Orencole, S. F., C. A. Dinarello [1989] Cytokine 1:14). For murine lymphocytes, two distinct CD4 expressing helper T cell subsets, Th1 and Th2, have been identified which differ in terms of factor response and production. Strictly similar subsets of distinct human T lymphocytes have not yet been described. Murine Th2 T cells secret IL-10, a 17 kD molecule which can inhibit Th1 cell activity. IL-10 has extensive homology with an uncharacterized EBV gene, BCRF1 (Baer, R., A. T. Bankier, M. D. Biggin, P. L. Deininger, P. J. Farrell, T. J. Gibson, G. Hatfull, G. S. Hudson, S. C. Satchwell, C. Sequin, P. S. Tuffnell, B. G. Barrell [1984] Nature 210:207).

Several growth factors have been proposed to have stimulatory activity for EBV transformed cells. These include BCGF (Ambrus, J. L., A. S. Fauci [1985] J. Clin. Invest. 75:732), IL-1 (Blazar, B. A., L. M. Sutton, M. Strome [1986] Canc. Immunol. 22:62), CD23 (Swendeman, S., D. A. Thorley-Lawson [1987] EMBO J. 6:1634), an unidentified lymphokine, called autocrine B Growth Factor, aBGF (Buck, J., U. Hammerling, M. K. Hoffman, E. Levi, K. Welte [1987] J. Immunol. 138:2923), and, most recently, IL-6 (Muraguchi, A., H. Nishimoto, N. Kawamura, A. Hori, T. Kishimoto [1986] J. Immunol. 137:179). Thus far, none of these molecules has been shown to be universally present or absolutely required for growth of EBV positive lymphoblastoid cell lines. BCGFs of 25-30 kD and 60 kD, which are similar to T cell derived lymphokines, have been identified in supernatants from EBV-carrying cells. 60 kD BCGF has been purified to homogeneity and, although an activator of stimulated normal B cells, it is not produced by all EBV-carrying lines or even by all cells in individual secretor lines. BCGF production has also been reported for activated normal B lymphocytes (Muraguchi et al., supra).

Certain EBV-carrying cells have been reported to function as antigen presenting cells, contain IL-1 like activity in their supernatants, and express mRNA for IL-1. One laboratory reported the purification of a novel IL-1 from an EBV-carrying line (Bertoglio, J., J. Dosda, R. Stancou, E. Wollman, D. Fradelizi [1989] J. Mol. Cell Immunol. 4:139) but later revised their findings (Bertoglio, J., E. Wollman, A. Shaw, L. Rimsky, D. Fradelizi [1989] Lympho. Research 8:19). This same laboratory now reports that an IL-1-like activity (ADL) is elicited by a 12 kD protein produced by both EBV and HTLV-1 transformed cells (Wakasugi, H., N. Wakasugi, T. Trusz, Y. Tagaya, J. Yodoi [1989] J. Immunol. 142:2569; Tagaya, Y., Y. Maeda, A. Mitsui, N. Kondo, H. Matsui, J. Hamuro, N. Brown, K. Arai, T. Yokota, H. Waksugi, J. Yodoi [1989] EMBO J. 8:757). Cloning of this protein, ADL, indicates it is a member of the human thioredoxin family with no direct relatedness to IL-1, although it may enhance IL-1 functions. Vigorous attempts by our laboratory using both Northern blotting and reverse transcription polymerase chain reaction (RT-PCR) demonstrate clearly that neither IL-1α nor IL-1β is expressed by these NAD-20 cells.

CD23, originally identified as a 45 kD differentiation antigen on EBV-infected cells, is expressed on all activated human B cells and macrophages. CD23 is identical to the low affinity Fc receptor for IgE (FcₑR11/CD23) (Detrance, T., J. P. Aubry, F. Rousset, B. Vandervliet, J. Y. Bonnefoy, N. Arai, Y. Takebe, T. Yokota, F. Lee, K. Arai, J. de Vries, J. Banchereau [1987] J. Exp. Med. 165:1459). A soluble 25 kD form of CD23 is shed into cell supernatants. At present, there is much interest in CD23 because the latent EBV genes, EBNA 2 and LMP, appear to induce its expression in B lymphocytes (Wang, F., C. D. Gregory, M. Rowe, A. B. Rickinson, D. Wang, M. Birkenbach, H. Kikutani, T. Kishimoto, E. Kieff [1987] Proc. Natl. Acad. Sci USA 84:3452). Some reports have suggested that it is a receptor for the low molecular weight BCGF (Gordon, J., A. Webb, G. R. Guy, L. Walker [1986] Eur. J. Immunol. 16:1627), and a shed form has been reported to function as an autocrine growth factor (Swendeman et al., supra). Conflicting reports, however, have also appeared. Recombinant shed FcₑR11/CD23 did not stimulate B cell proliferation, whereas it did bind IgE (Uchibayashi, N., H. Kikutani, E. L. Barsumian, R. Hauptmann, F. -J. Schneider, R. Schwendenwein, W. Sommergruber, W. Spevak, I. Mourer-Fogy, M. Suemura, T. Kishimoto [1989] J. Immunol. 142:3901). Highly purified shed CD23 from supernatants failed to stimulate B cell growth, and CD23 on the plasma membrane was not demonstrated as the receptor for the low molecular weight BCGF.

Acquisition of an autocrine growth cycle, whereby a cell both secretes and responds to endogenous growth stimulating factors, may be one means by which cancer cells achieve autonomy. Normal B lymphocytes transformed by EBV and malignant cell lines containing the EBV genome have been found to produce autostimulatory growth factors (Blazar, B. A., L. M. Sutton, M. Strome [1983] Can. Res. 43:4562).

### BRIEF SUMMARY OF THE INVENTION

The subject invention concerns the discovery and purification of a novel secreted autostimulatory factor from an EBV-carrying lymphoblastoid cell line. The novel protein has been named IL-X. IL-X, which can be isolated by size exclusion HPLC on TSK-SW-3000 columns, migrates at an apparent molecular weight of approximately 42 kD in SDS-polyacrylamide gels. Thus, this protein is referred to herein as the 42 kD IL-X protein, but it should be recognized that, due to the nature of SDS-PAGE analysis, the molecular weight of the protein may deviate slightly from the 42 kD value. IL-X stimulates proliferation of both EBV-carrying B lymphocytes and CON A activated normal T lymphocytes, suggesting a diverse role in control of lymphocyte proliferation. Growth of EBV-carrying lymphoblastoid cells is significantly reduced by antibody directed against either native IL-X or IL-X derived peptides (p-IL-X).

IL-X, either alone or in combination with other immunoregulatory molecules, can contribute to the establishment of long term lines of normal B cells in vitro. Such lines can also be used to generate monospecific human antibodies.

Severe pathology resulting from the uncontrolled proliferation of EBV-transformed B cells occurs in neoplasia and fatal infectious mononucleosis, as well as in situations of immunosuppression, immunodeficiency, and AIDS. Antibodies that neutralize IL-X activity, antisense DNA, or other antagonists of IL-X can be used to inhibit IL-X bioactivity or IL-X receptor function. For example, antibodies or other antagonists can be used to interrupt autocrine loops established in B cell neoplasia. Alternatively, exogenous replacement of IL-X can ameliorate certain B cell or T cell immunodeficiencies.

### BRIEF DESCRIPTION OF THE DRAWINGS

FIG. 1 shows the effect of size exclusion HPLC column fraction 18 (comprising IL-X) from the separation of NAD-20 medium and RPMI control medium on growth of NAD-20 cells (10³ well) in ³H-thymidine assays. Estimate of picograms of IL-X is determined from silver stained SDS-PAGE gels with a sequentially diluted standard.

FIG. 2 shows effect of HPLC column fractions from NAD-20 medium and RPMI control on CON A-stimulated D10.G4.1 cells (10^{4/}well). Estimate of picograms IL-X is determined by silver stained SDS-PAGE gels with sequentially diluted standard.

FIG. 3 shows mean growth of NAD-20 cells enumerated in triplicate cultures on day 4 following the addition of 5% rabbit serum. Differences in growth between the NRS (normal prebleed) serum and the anti pIL-X serum and between the NRS and the anti IL-X serum are significant (p<0.05).

### BRIEF DESCRIPTION OF THE SEQUENCES

SEQ ID NO. 1 is tryptic fragment A from IL-X

SEQ ID NO. 2 is tryptic fragment B from IL-X.

SEQ ID NO. 3 is tryptic fragment C from IL-X.

SEQ ID NO. 4 is a hypothetical 10-amino acid fragment corresponding to reported positions 2-11 of tryptic fragment A.

SEQ ID NO. 5 is a 48-mer oligonucleotide probe that can be used according to the subject invention.

SEQ ID NO. 6 is the RT-PCR fragment from IL-X

SEQ ID NO. 7 is a DNA sequence obtained by a reverse transcriptase-polymerase chain reaction procedure.

### DETAILED DISCLOSURE OF THE INVENTION

We have isolated a novel molecule (IL-X) with an apparent molecular weight of 42 kD from an EBV-carrying lymphoblastoid cell line. Analysis of the amino acid sequence of three peptides obtained by tryptic digest of IL-X indicates that this molecule has not been previously described. We have found that IL-X augments proliferation of both B and T lymphocytes, suggesting broad immunoregulatory effects for this protein. Also, we have discovered that growth of EBV-transformed human B lymphocyte lines is reduced by either antibody to IL-X or antibody to an IL-X peptide. As described herein, peptide sequences can be used to construct oligonucleotides to screen cDNA libraries for IL-X cDNA. Recombinant protein can be used for functional studies and for the preparation of monospecific antisera

The novel interleukin compounds of the subject invention have a variety of uses. For example, IL-X, or fragments or derivatives thereof, can be used to raise antibodies as described herein. Also, IL-X, or fragments or derivatives thereof, can be used to enhance the growth of cells in vitro. In one preferred embodiment of the subject invention, an IL-X protein can be used to grow primary human B cell cultures. This has not previously been possible. The capacity to grow normal B cell cultures will facilitate investigation of B cell biology and growth regulation in non-transformed cells. Thus, an IL-X protein can be added to primary human B cell cultures to facilitate proliferation of these cell lines. Such cell lines can also be used advantageously in the production of human monoclonal antibodies.

The IL-X proteins can also be used in therapeutic or prophylactic situations. Such situations would include, for example, disease states associated with diminished levels of IL-X. For example, IL-X may be administered to enhance B cell or T cell proliferation to alleviate conditions associated with diminished B cell or T cell populations.

As would be readily appreciated by one skilled in this art, the IL-X proteins of the subject invention can also be used as molecular weight standards, or as inert proteins in an assay. The polypeptides can also be used to detect the presence of antibodies that are immunoreactive with IL-X.

A further utility of the subject invention concerns the development of immunological reagents to recombinant IL-X (rIL-X) for diagnosis and treatment. Antibodies raised to an IL-X protein can have a variety of uses. For example, such antibodies may be used for purification or identification of IL-X. Also, such antibodies may be useful in therapeutic or prophylactic applications where it is desired to inhibit the activity of IL-X Such situations would include, for example, disease states associated with increased levels of IL-X or associated with excessive B or T cell proliferation, including those disease states mediated by the Epstein-Barr virus. This could include neoplasia, B cell lymphomas, and cases of severe mononucleosis.

IL-X activity may also be inhibited by the administration of soluble IL-X receptors that would bind with IL-X and inhibit its normal biological activity. Alternatively, analogs of IL-X could be designed that would bind to IL X receptors without activating those receptors. In yet another example, antibodies to the receptors could be administered. Such antibodies could sterically block the receptors, thereby preventing binding by IL-X. Other methodologies and variations of these methodologies would be apparent to one skilled in the art.

A further method for inhibiting the activity of IL-X involves the administration of antisense DNA that would specifically bind to the DNA that encodes IL-X Such an administration of antisense DNA would block the production of IL-X, thereby reducing or eliminating its biological activity. The antisense DNA can be administered by techniques known in the art, including encapsulation in liposomes.

The administration of IL-X proteins or antibodies to IL-X would be in accordance with standard techniques and formulations useful for administering such proteins and antibodies. In one embodiment, the IL-X proteins or anti-IL-X antibodies may be conjugated to antibodies or receptors. These antibodies or receptors could then direct the IL-X biological activity to a desired location in the body.

As used herein, reference to IL-X refers to the 42 kD protein specifically exemplified herein, as well as fragments, precursors, analogs, or derivatives of that 42 kD protein that retain biological or immunological characteristics associated with the 42 kD protein.

A further aspect of the invention pertains to polynucleotide sequences which encode IL-X proteins. The polynucleotide sequences of the subject invention may be composed of either RNA or DNA Preferably, the polynucleotide sequences are composed of DNA. A full length gene encoding the 42 kD protein and any precursor can be obtained as described herein. Also, a person skilled in the art could readily synthesize DNA sequences encoding IL-X peptides. Specific examples of IL-X peptides are provided herein. The nucleotide sequences encoding the peptides could be used to produce recombinant peptides, or they could be used as probes or as primers for diagnostic and/or analytical PCR procedures. The polynucleotides of the subject invention can also be used as DNA or RNA sizing standards. Methods for obtaining full length genes are described in greater detail below.

As used herein, the term “isolated” refers to obtaining a protein or antibody in a form other than that which occurs in nature. This may be, for example, obtaining IL-X by purifying crude samples, or it could be the recovery of recombinant IL-X from transformed cell lines. In the case of polynucleotide sequences, “isolated” would mean, for example, that the sequence is no longer associated with other DNA sequences with which it would naturally occur. Thus, the claimed polynucleotide sequence may be placed into a plasmid or other vector or transformed or transfected into the genome of a host. In the case of antibodies, “isolated” refers to antibodies which, through the hand of man, have been produced or removed from their natural setting. Thus, isolated antibodies according to the subject invention would include antibodies raised as the result of purposeful administration of IL-X compounds to an appropriate host.

Screening of EBV-carrying B lymphocyte cDNA libraries.

The IL-X gene can be cloned using oligonucleotide sequences derived from protein sequence either directly or using PCR primers to generate larger, non-degenerate probes. As suitable protein sequence data have been obtained, one can screen available cDNA libraries derived from both human and non-human mammalian EBV-carrying lymphoblastoid cell lines for IL-X sequences. Degenerate oligonucleotide pools or longer (40-50-mers) single sequence “guessmers” with or without inosine substitutions can be synthesized with the help of codon preference tables from the tryptic peptide amino acid sequence data provided herein. This approach is successful in providing IL-X cDNA clones as evidenced by the primary filter screening of a bacteriophage λgt11 library made from the tamarin cell line, BLCL-100-75, with a degenerate oligonucleotide as described in the examples which follow. Six hybridizing plaques from an initial plating of 10⁶ recombinant phage have been plaque purified. The fidelity of these “positives” can be evaluated by (1) re-hybridization to another oligonucleotide probe synthesized to compare to protein sequence derived from a different tryptic fragment of IL-X; and (2) identification of nucleotide sequences that code for the IL-X peptide sequences. If a non-human cDNA for IL-X is isolated, it could be used as a probe for obtaining the human clone.

Human EBV-LCL derived cDNA libraries can be commercially made using either the reliable λgt11 vector or possibly one of the recently developed directional cloning derivatives (e.g., λDR2, lambdaZAP). The vendor (Clontech, Stratagene) can be supplied with poly(A)⁺RNA extracted by standard procedures known to one skilled in the art from EBV-transformed NAD-20 cells expressing IL-X bioactivity and a demonstrable 42 kD IL-X polypeptide. To verify the presence of IL-X transcripts in these poly(A)⁺RNA samples, one can use reverse transcription-PCR (RT-PCR) with combinations of antisense and sense strand oligonucleotides derived from different IL-X tryptic fragment amino acid sequence data. The optimal conditions for primer annealing can be assessed using mRNA extracted from both expressing and non-expressing cell lines. Similarly, the best conditions for oligonucleotide probe hybridization can be evaluated using dot-blots and/or Northern blots of the same samples if sufficient RNA is obtained. Ideally, the same samples that test positive for RT-PCR and/or blot hybridization should be used for library construction. The successful generation of a reproducible PCR fragment that itself cross-hybridizes on Southern blots with other ³²P end-labelled oligonucleotides has the added advantage of providing a more specific IL-X probe for library screening than straight degenerate oligonucleotides.

Standard methods can be used to screen the bacteriophage library. Duplicate lifts onto 150 mm nylon membranes (e.g., GeneScreen, Hy-Bond) from 20 plates containing 50,000 plaques each can be used in the initial screening using ³²P-labelled PCR fragments (amplification labelled) or oligonucleotides (kinase 5′ end-labelled). Several rounds of replating and re-hybridization of “positive” plaques leads to the isolation of bona fide cDNA clones encoding IL-X.

DNA sequencing of IL-X cDNA.

To facilitate restriction mapping and DNA sequencing, the isolated cDNAs can either be sub-cloned into a more convenient plasmid vector such as pBluescript (Stratagene) or generated as plasmids directly depending upon the vector of origin. The cDNA inserts of hybridizing clones can be amplified directly from either plug-eluted phage or colony lysates by PCR. Primers complementary to lambda vector sequences flanking the cloning site and containing 5′ terminal rare restriction enzyme recognition sequences convenient for sub-cloning have been synthesized and successfully used. PCR amplification also provides a rapid screen for the longest cDNA clones to be sub-cloned. Plasmid DNA minipreps from pBluescript sub-clones of IL-X cDNAs can be used for preliminary restriction enzyme mapping or direct sequencing of 5′ and 3′ ends using the dideoxynucleotide chain A terminator method with flanking vector sequence primers. The complete sequence of a selected clone is readily obtained by generating a nested series of overlapping deletion sub-clones from the parent plasmid using the exonuclease EI-mung bean nuclease method (Stratagene protocol), or by primer walking using oligonucleotides synthesized from sequence data.

Expression of the IL-X cDNA and further purification of IL-X protein.

IL-X protein encoded by full-length cDNA clones isolated from the λgt library can be expressed in E. coli as a β-galactosidase fusion protein following IPTG induction. Available IL-X antibodies can be used to screen the library for an in-frame clone by standard procedures. Bacterial cell lysates can be used to raise crude polyclonal antibodies in rabbits and rats and adsorbed against lysates from non-recombinant E. coli host cells yielding antisera relatively specific for IL-X This antibody can serve as a preliminary reagent in Western blot analysis of IL-X until highly purified protein can be obtained to use in the production of monoclonal and mono-specific polyclonal antibodies. This antibody therefore enables one to determine whether IL-X is expressed exclusively in cells of the B lineage. Additionally, by use of crude lysates in growth assays with EBV-ECL, one can determine whether the cDNA clones selected encode an active factor.

Once the fidelity of an IL-X clone is established, then large-scale expression of recombinant protein can be undertaken using, for example, a baculovirus expression system. The IL-X cDNA insert can be sub-cloned into a modified form of the original Summers (Lukow, V. A., M. D. Summers [1989] Virology 170:31) baculovirus transfer vector pVL1392. The modification to this vector system is the addition of six tandem histidine residues either at the amino or carboxyl-terminus of the expressed protein to facilitate one-step purification by binding to immobilized Ni²⁺ affinity columns (Qiagen, InVitrogen Corp.). The recombinant IL-X isolated in this fashion can be used directly for antibody production. If desired, the histidine sequence can be removed by enzymatic cleavage using a rare protease-sensitive site situated at the junction of the histidine tag and the N-terminus of the encoded protein. Throughout the purification scheme, the location of functional recombinant IL-X can be evaluated both by addition to growth assays with EBV-LCL and by Western blot analysis after SDS-PAGE of extracts and column fractions.

In addition, recombinant IL-X radiolabelled with ³⁵S-methionine for receptor binding assays, described below, can be produced by metabolic labelling of Sf9 insect cells infected with IL-X recombinant virus and grown in Excel met—medium (JRH Biosciences) supplemented with ³⁵S-methionine. Alternatively, very high specific activity protein can be obtained by in vitro translation of transcripts containing plant viral 5′ leader sequences (Jobling, S. A., L. Gehrke [1987] Nature 325:622-625). Construction of the appropriate transcription clone in pBluescript can be achieved by insertion of the AMV leader sequence oligonucleotide upstream and in-frame with the IL-X coding region. Large amounts of fusion transcript for use in reticulocyte lysate translations can then be generated with either T3 or T7 RNA polymerase directly from linearized template of such a pBluescript sub-clone. Using the IL-X construct, large quantities of high specific activity IL-X can be produced in vitro.

Production of monospecific antibodies.

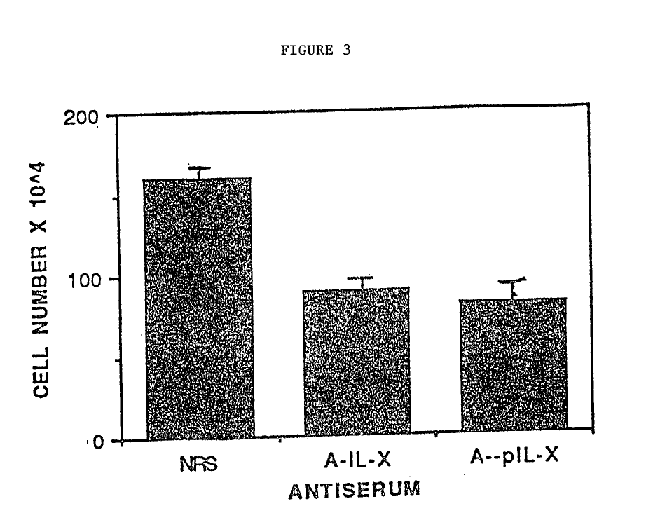

As described herein, two rabbit antisera have been raised to IL-X One serum was induced by IL-X obtained from SDS-PAGE gels; the second serum was induced by a KLH coupled 18 amino acid peptide (pIL-X) synthesized to correspond with an internal amino acid sequence from IL-X. Both antisera react with native IL-X on dot blots. In experiments with NAD-20 cells, these same sera interfered with cell growth (FIG. 3). Both of these antibodies can be used for the examples outlined below.

Additional antibodies to IL-X can be made using, for example, the following immunogens:

(i) The IL-X peptide (pIL-X) which is recognized on the native IL-X protein by rabbit anti-pIL-X; this antiserum can be made in rats and enables the development of a specific ELISA for IL-X. Alternatively, the presently available rabbit anti-IL-X could be biotinylated and used directly for ELISA development.

(ii) Synthesized peptides prepared from the other sequenced tryptic fragments of IL-X; this antiserum can be made in rabbits or other animals.

(iii) Recombinant IL-X; this antiserum can be made in rabbits or other animals and can provide a more specific reagent for the entire IL-X molecule than the antiserum raised against IL-X from SDS-PAGE gels.

Positive heterosera can be affinity purified by column chromatography with the synthesized peptide or on a microscale using nitrocellulose strips containing IL-X. Identity of IL-X material can be established by ELISA, immunoprecipitation, and/or immunoblot by standard techniques. Antisera produced can be evaluated for specificity with appropriate cell and lysate controls and can be tested for reactivity against other known cytokines.

Monoclonal antibodies can be produced against IL-X to obtain even greater specificity, although the raising of monoclonal antibodies is not essential to these experiments. ELISAs, used to screen hybridomas for antibody production, can be performed also with recombinant or HPLC-purified IL-X Antibody to mouse IgG coupled to alkaline phosphatase can be used as a probe. In addition, monoclonal antibodies can be raised by standard techniques to small peptides synthesized to gm correspond to amino acid sequences obtained from tryptic fragments of IL-X such as pIL-X.

Following are examples which illustrate procedures, including the best mode, for practicing the invention. These examples should not be construed as limiting. All percentages are by weight and all solvent mixture proportions are by volume unless otherwise noted.

### EXAMPLE 1

Biochemical Purification of IL-X

IL-X can be purified from supernatants of the NAD-20, EBV-positive lymphoblastoid cell line by size exclusion HPLC on TSK-3000 gel filtration columns. NAD-20 cells are freely available and can be obtained, for example, from Dr. George Klein, Department of Tumorbiology, Karolinska Institute, Stockholm, Sweden.

In addition, the NAD-20 cell line has been deposited with the American Type Culture Collection (ATCC), 12301 Parklawn Drive, Rockville, Md. 20852 USA, on Dec. 8, 1993. The culture has been assigned accession number CRL 11501 by the ATCC.

The subject culture has been deposited under conditions that assure that access to the culture will be available during the pendency of this patent application to one determined by the Commissioner of Patents and Trademarks to be entitled thereto under 37 CFR 1.14 and 35 U.S.C. 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least thirty (30) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

Concentrated supernatants were prepared from the NAD-20 line by a 24 hour incubation of logarithmically growing cells in RPMI, followed by passage through an Amicon stir cell containing a Spectrapore ultra filter with a 1000 MWCO pore size. Following dialysis, concentrated supernatant applied to the column was eluted with phosphate buffered saline (PBS) and monitored at an absorbance of 280 nm. Four-mililiter fractions were collected and tested for activity. The B lymphocyte growth stimulatory activity was recovered in fractions 18-20 with an occasional trailing peak at fraction 22. Column fractions were concentrated 10 times and processed on SDS-PAGE gels. Only one band, with an apparent molecular weight of 42 kD, was detectable by silver stain. The molecular weight appeared to be identical in size when electrophoresis was performed under both reducing and nonreducing conditions.

Purification of IL-X by HPLC results in a substantial removal of this protein from concentrated supernatants. Approximately 40% of the growth stimulatory activity in unfractionated supernatants is recovered in the active HPLC fractions. In Table 1, data are presented for one purification of IL-X. Values for specific activity may be inaccurate, as silver stain is imprecise for this purpose.

TABLE 1
Purification of IL-X

| | Protein | Stimulatory activity | | Specific | |
|---|---|---|---|---|---|
| | content*** | Experi- | CPM/ | activity | |
| Source | ng/μl | mental** | μl | (activ/ng) | Units/mg* |
| NAD-20 | 0.04 | 1429/100 μl | 14.29 | 357.3 | |
| medium | | | | | |
| HPLC | 0.002 | 917/12.5 μl | 73.46 | 36,680.0 | 3.7 × 10⁸ |
| Frac-18 | | | | | |
| NAD-20 | | | | | |

***estimate based on silver stained SDS-PAGE gel
**average CPM over control
*1 unit is defined as causing a 100% increase in ³H-thymidine uptake

Although the apparent molecular weight of IL-X, 42 kD, indicates that IL-X is larger than most immunomodulatory molecules, we have in addition demonstrated by functional assay and mRNA analysis that IL-X is not IL-1α or IL-1β (mRNA), IL-2 (CTLL2 cells), IL-3 (DA1 cells), IL-4 (monocyte CD23 induction), IL-5 (Western blot using antibody from Genetics Institute), TNFα (ELISA, Cistron Biotechology, Inc.), or IL-6 (B9 cells).

The 42 kD IL-X is not found in culture supernatants of macrophage, T lymphocyte, and epithelial cell lineages, whereas supernatants from 4 different EBV-carrying cell lines contain a molecule of the same size as IL-X.

### EXAMPLE 2

Molecular Characterization of IL-X

IL-X was purified from concentrated supernatants by SDS-PAGE and electrophoretically transferred onto nitrocellulose. The nitrocellulose was stained with Ponceau S and the appropriate band was excised. This band on the nitrocellulose was incubated with PVP-40 and digested with trypsin. At this stage tryptic fragments could be resolved by reverse-phase HPLC. The IL-X used for purification was obtained from an SDS-PAGE gel and selected by molecular weight. As IL-X is the same apparent molecular weight as actin, the most abundant protein in cells, we felt it was necessary to first select tryptic fragments for sequencing which could not contain any possible actin contaminants. Concentrated NAD-20 medium and purified actin were individually separated by SDS-PAGE, transferred to nitrocellulose, and digested with trypsin in parallel. The tryptic fragments from IL-X and actin were processed by reverse phase HPLC in an identical manner. A comparison of the peptide maps indicates that there are several tryptic fragments unique to IL-X that are not similar to any tryptic fragments found in actin. Tryptic fragments unique to IL-X were sequenced. Three HPLC fractions containing well-separated fragments were transferred to PVDF membrane and sequenced in a gas phase sequenator.

Amino acid sequence of 3 tryptic fragments from IL-X.

The following sequences were determined:

Tryptic fragment A:

[SER], U-E, GLY, VAL, ALA, GLU, ASN, -, PHE, (GLY), ASN (SEQ ID NO. 1)

Tryptic fragment B:

TYR, ASN, PRO, HIS, VAL, ILE, LEU, SER, VAL, ALA, GLY, PRO, ALA, THR, PHE, GLU, THR (SEQ ID NO. 2)

Tryptic fragment C:

VAL, VAL, ALA, SER, PHE, GLY, GLY, GLY, ALA, PHE, PRO, GLY, VAL, THR, THR, PHE, ASN, GLU, GLY, PHE, (ASN), (GLY) (SEQ ID NO. 3)

Because of the ambiguity of positions 1, 8, and 10 of tryptic fragment A, the following hypothetical 10 amino acid sequence generally corresponding to positions 2-11 of fragment A was used for comparison with reported molecules to overcome any ambiguity:

ILE, GLY, VAL, ALA, GLU, ASN, any aa, PHE, any aa, ASN (SEQ ID NO. 4)

The sequences for tryptic fragments B and C were used for comparison purposes. A search in the most recent National Biomedical Research Foundation Protein Library, most recent GenBank Translated version, and the Swissprotein databank, the National Center for Biotechnology Information (NCBI) Peptide Sequence database, and the latest Entrez release (6.0) using the BLAST Internet services, found neither identity nor similarity for tryptic fragments A, B, or C with any reported sequences.

The amino acid sequence of an IL-X peptide fragment derived from a reverse-transcriptase-polymerase chain reaction (RT-PCR) product was also determined. The RT-PCR product has the DNA sequence shown in SEQ ID NO. 7. The RT-PCR product was obtained using synthetic degenerate oligonucleotide primers based on tryptic peptide sequences A, B, and C.

A 48-mer sense-strand oligonucleotide (termed C+) was made based upon the amino acid sequence of tryptic peptide C using the neutral base inosine at positions of 4-fold, third position degeneracy in the genetic code. Two anti-sense, 64-fold degenerate 18-mers (A− and B−) were also synthesized based upon the amino acid sequences of the IL-X tryptic fragments A and B, respectively. These anti-sense oligonucleotides were each used to prime NAD-20 cellular RNA in a reverse transcriptase driven first strand cDNA reaction, followed by PCR amplification using the sense strand C+ 48-mer. RT-PCR of NAD-20 RNA using primers C+ and A− yielded an ≈480 bp fragment (CA), whereas a combination of primers C+ and B− on the same template generated a product CB of ≈220 bp in length. The 220 bp CB fragment could also be re-amplified using the CA fragment as the template primed with the oligonucleotides B− and C+, whereas oligonucleotides A− and B− produced no PCR product in the same reaction. Based upon these PCR reactions, we inferred the order and spacing of peptides in IL-X to be C, B, A Complete sequencing of the CA fragment after direct subcloning into the T/A plasmid pCRII (InVitrogen Corp.) yielded a 498 bp nucleotide sequence that encodes all three tryptic peptides (C, B, and A) in frame within a 166 amino acid portion of the IL-X protein.

IL-X RT-PCR Fragment:

Phe Gly Gly Gly Ala Phe Pro Gly Val Thr Thr Phe Asn Glu Gly Phe Ala Lys
Gly Ile Leu Tyr Tyr Asn Gln Lys His Lys Ser Ser Lys Ile Tyr His Thr Ser
Pro Val Lys Leu Asp Ser Gly Phe Thr Ala Gly Glu Lys Met Aen Thr Val Ile
Asn Asn Val Leu Ser Ser Thr Pro Ala Asp Val Lys Tyr Asn Pro His Val Ile
Leu Ser Val Ala Gly Pro Ala Thr Phe Glu Thr Val Arg Leu Ala Asn Lys Gly
Gln Tyr Val Ile Gly Val Asp Ser Asp Gln Gly Met Ile Gln Asp Lys Asp Arg
Ile Leu Thr Ser Val Leu Lys His Ile Lys Gln Ala Val Tyr Glu Thr Leu Leu
Asp Leu Ile Leu Glu Lys Glu Glu Gly Tyr Lys Pro Tyr Val Val Lys Asp Lys
Lys Ala Asp Lys Lys Trp Ser His Phe Gly Thr Gln Lys Glu Lys Trp Ile Gly
Val Ala Glu Asn

The amino acid sequences corresponding to tryptic peptides C, B, and A are shown in bold letters, above, and are located within the RT-PCR peptide fragment The sequence is given beginning at the amino terminal end of the peptide fragment. The RT-PCR peptide fragment shows no significant matches or degree of homology to any proteins or polypeptides currently contained in any NCBI database. Furthermore, the RT-PCR cDNA fragment also lacks homology with any nucleotide sequence in the NCBI and Entrez nucleotide sequence database. Accordingly, the above amino acid sequences are concluded to be unique, and belong to a novel approximately 42 kD protein designated IL-X.

### EXAMPLE 3

Purified IL-X Enhances B Cell Growth

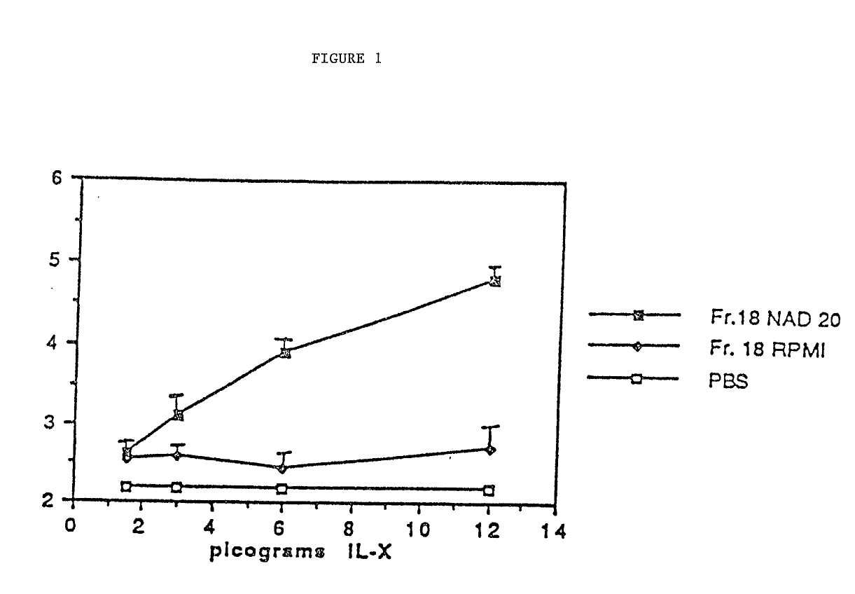

The column fractions containing detectable IL-X enhance growth of NAD-20 cells as determined by ³H-thymidine incorporation. NAD-20 cells were incubated at low density to arrest their growth rate before use in these assays. Results of one representative experiment with HPLC fraction 18 are presented in FIG. 1. HPLC column fraction 18, which contained the greatest amount of material on SDS-PAGE, also exhibited the greatest amount of activity in B lymphocyte growth assays compared to HPLC fractions.

Studies with rabbit antibody to rIL-1α and rabbit antibody to rIL-1β demonstrated that these antibodies did not significantly reduce the enhancement of B cell growth by IL-X.

### EXAMPLE 4

Further Characterization of Biological Activity of IL-X

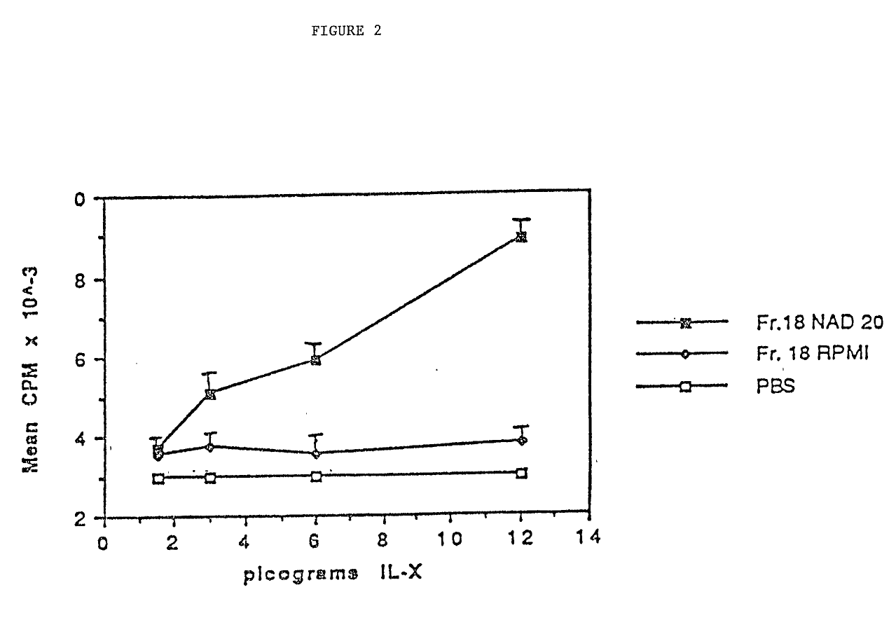

Unfractionated supernatants from EBV-carrying lymphoblastoid cell lines contain growth stimulatory activity for human T lymphocytes. These supernatants enhance proliferation of CON A activated human T lymphocytes. They also are active in assays with PHA activated murine thymocytes. These same supernatants do not contain IL-2 as indicated by their inability to support growth of CTLL 2 cells. The HPLC column fractions which contained detectable IL-X costimulated the proliferation of the CON A murine T cell clone, D10.G4.1. D10.G4.1 is an IL-2 dependent helper T cell which is commonly used to assay for the comitogenic effect of IL-1. Results of one representative experiment with HPLC fraction 18 are presented in FIG. 2. As in the case of IL-1, human IL-X appears to enhance lymphoid cell growth without species restriction.

### EXAMPLE 5

Demonstration that IL-X Does Not Enhance the Growth of an Epithelial Cell Line

Neither the unfractionated IL-X containing supernatant nor HPLC fraction 18 enhanced growth of H-135, a colon carcinoma cell line. This suggests that IL-X may exhibit some specificity as a regulatory molecule.

### EXAMPLE 6

Development of Antibodies to IL-X

(1) Antibodies to entire IL-X molecule.

IL-X, separated by SDS-PAGE from concentrated (4000 X) NAD-20 supernatants, was cut out of gels. Gel slices were ground, emulsified in Freund's adjuvant, and injected subcutaneously into a rabbit. Following subsequent immunizations, rabbit antiserum to IL-X (anti-IL-X) was prepared by standard techniques. Rabbit anti-IL-X recognizes the native IL-X protein on dot blots and SDS-PAGE processed IL X protein on Western blots.

(2) Antibodies to IL-X peptide.

An 18-amino acid peptide corresponding with the first 18 residues of the sequence for tryptic fragment C reported above was synthesized. This peptide was conjugated with KUH via cysteine and used to immunize a rabbit. This rabbit antiserum (anti-pIL-X) recognized the unconjugated peptide and the native IL-X protein on dot blots. The anti-pIL-X serum, however, does not recognize processed IL-X following SDS-PAGE and Western blotting techniques. Western blots probed with either enzyme or ¹²⁵I conjugated goat anti-rabbit IgG were equally unsuccessful in demonstrating any binding of the anti-pIL-X antibody to IL-X.

As is known in the art, the techniques of SDS-PAGE and Western blotting cause antigen denaturation, and only denaturation-resistant epitopes on molecules can be recognized by antibodies. The inability to recognize epitopes destroyed by denaturing reagents is commonly experienced with monoclonal antibodies which bind with only one epitope. The epitope(s) on IL-X recognized by the anti-pIL-X serum has as its greatest possible dimension 18 residues of primary amino acid sequence and is likely to be considerably smaller due to possible intramolecular secondary and tertiary structure. In these Western blots, our rabbit anti-pIL-X functions more like a monoclonal than a polyclonal serum.

### EXAMPLE 7

Demonstration that Antibody to IL-X Reduces B Cell Growth

In order to further evaluate the proliferative effect of IL-X on B lymphocytes, the rabbit antibodies to IL-X described above were added to growing B lymphocyte cultures. Growth of NAD-20 cells was significantly reduced, compared to growth in the presence of normal prebleed rabbit serum, by the addition of either rabbit anti-IL-X or by rabbit anti-pIL-X. Results from one preliminary experiment illustrating the mean number of cells after the addition of either control, anti-IL-X, or anti-pIL-X serum are presented in FIG. 3. The reduction of cell growth by specific antisera to IL-X provides additional proof of a role for IL-X in the growth of EBV-carrying B lymphocytes.

### EXAMPLE 8

Screening of EBV-Carrying Tamarin cDNA Library

An EBV-carrying tamarin B lymphocyte cDNA library (a bacteriophage λgt11 library made from the tamarin (Sanquinis oedipus) cell line, BLCL-100-75) was screened. Screening was accomplished with a degenerate oligonucleotide synthesized to correspond with the amino acid sequence for tryptic fragment C, described above. The sequence of this oligonucleotide probe is as follows:

| | |
|---|---|
| 5′-TTY GGI GGI GGI GCI TTY CCI GGI GTI ACI ACI TTY AAY GAR GGI TTY-3′ | (SEQ ID NO.5) |

This 48-mer with ten inosine substitutions was kinase end-labelled with ³²p and hybridized (under conditions of low stringency, i.e., hybridization at 25° C. and washing at 30° C. with 6×SSC) to six plaques from an initial plating of 10⁶ recombinant phage. These clones have been plaque purified (3 rounds). Preliminary analysis of the cDNA inserts by PCR amplification using λgt11-specific primers (carrying convenient restriction sites for subcloning) flanking the EcoRI cloning site has shown cDNA inserts in the range of 1.9 kb in two of these six clones. These two clones can be sub-cloned into M13 and/or plasmid vector for dideoxynucleotide sequencing to generate structural data on IL-X.

### EXAMPLE 9

IL-X Receptor Analysis

Recombinant IL-X can be radiolabeled either by iodination (Bolton-Hunter Reagent, New England Nuclear) or in vitro translation and checked for the maintenance of biological activity in functional assays of growth of EBV-carrying B lymphoblastoid cell lines. Radiolabelled IL-X can be incubated with B lymphocytes for 1 hour. Cells can be centrifuged through 8% w/v silicon fluid (SF1250) and the pellets evaluated for bound radioactivity. Non-specific binding can also be determined. B lymphocytes, normal resting and activated, as well as virus transformed B lymphocytes, and B lymphocytes of malignant origin, can be evaluated for the expression of a receptor for IL-X If binding is demonstrated, the number of expressed receptors can be evaluated by Scatchard analysis.

If a cell line with high level of specific IL-X receptor binding is identified, poly(A)⁺RNA extracted from this cell line can be used to make a cDNA mammalian expression library (e.g., pcDM8 or pcDNAI from InVitrogen). Plasmid DNA, first from the total library and then progressively smaller sub-pools, can be used to transfect monkey COS cells. The binding of radiolabelled IL-X to COS cells can be used to assay for the pressure of IL-X receptor and therefore lead to the isolation of an IL-X receptor.

It should be understood that the examples and embodiments described herein are for illustrative purposes only and that various modifications or changes in light thereof will be suggested to persons skilled in the art and are to be included within the spirit and purview of this application and the scope of the appended claims.

1. A purified IL-X polypeptide, comprising at least one amino acid sequence selected from the group consisting of SEQ ID NO: 1, SEQ ID NO: 2, SEQ ID NO: 3, and SEQ ID NO: 6.

2. The IL-X polypeptide of claim 1, comprising the amino acid sequence of SEQ ID NO: 6.

3. A purified IL-X polypeptide, having a molecular weight of approximately 42 kD as determined by SDS-PAGE or a fragment thereof sufficient to retain at least one biological activity selected from the group consisting of enhancing human B cell proliferation and enhancing human T cell proliferation, said polypeptide comprising at least one amino acid sequence selected from the group consisting of SEQ ID NO: 1, SEQ ID NO: 2, SEQ ID NO: 3, and SEQ. ID NO: 6.

4. The IL-X polypeptide of claim 1, said polypeptide having an approximate molecular weight of 42 kD as determined by SDS-PAGE.

5. The IL-X polypeptide of claim 3, said polypeptide comprising the amino acid sequence of SEQ ID NO: 6.

6. The IL-X polypeptide of claim 5, said polypeptide having an approximate molecular weight of 42 kD as determined by SDS-PAGE.


SEQUENCE LISTING (7 sequences)

SEQ ID NO: 1 - protein, 11 aa, not provided
SIGVAENXFGN

SEQ ID NO: 2 - protein, 17 aa, not provided
YNPHVILSVAGPATFET

SEQ ID NO: 3 - protein, 22 aa, not provided
VVASFGGGAFPGVTTFNEGFNG

SEQ ID NO: 4 - protein, 10 aa, not provided
IGVAENXFXN

SEQ ID NO: 5 - DNA (cDNA), 48 bp, not provided; single-stranded, linear
  misc_feature 6: /standard_name= “Inosine substitution” /label= i
  misc_feature 9: /standard_name= “Inosine substitution” /label= i
  misc_feature 12: /standard_name= “Inosine substitution” /label= i
  misc_feature 15: /standard_name= “Inosine substitution” /label= i
  misc_feature 21: /standard_name= “Inosine substitution” /label= i
  misc_feature 24: /standard_name= “Inosine substitution” /label= i
  misc_feature 27: /standard_name= “Inosine substitution” /label= i
  misc_feature 30: /standard_name= “Inosine substitution” /label= i
  misc_feature 33: /standard_name= “Inosine substitution” /label= i
  misc_feature 45: /standard_name= “Inosine substitution” /label= i
ttyggnggnggngcnttyccnggngtnacnacnttyaaygarggntty

SEQ ID NO: 6 - protein, 166 aa, not provided
FGGGAFPGVTTFNEGFAKGILYYNQKHKSSKIYHTSPVKLDSGFTAGEKMNTVINNVLSSTPADVKYNPHVILSVAGPATFETVRLANKGQYVIGVDSDQGMIQDKDRILTSVLKHIKQAVYETLLDLILEKEEGYKPYVVKDKKADKKWSHFGTQKEKWIGVAEN

SEQ ID NO: 7 - DNA, 498 bp, not provided; single-stranded, linear
tttggtggaggtgcattcccgggggtgacgacgtttaatgaggggtttgcaaaaggtattctatactacaaccaaaaacataaatcaagtaaaatttaccacacatcacctgttaaattagactcaggttttactgctggtgaaaaaatgaacactgttattaataatgttttatcttcaacaccagctgatgttaaatacaacccacatgttatcttatctgttgctggacctgctacatttgaaactgtaagattagcaaacaaaggtcaatatgtaattggtgttgactcagaccaaggcatgattcaagacaaagacagaattcttacatcagttctaaaacacattaaacaagctgtttatgaaacattattagatcttattcttgaaaaagaagaaggatataaaccatatgtagttaaagacaaaaaagcagacaaaaaatgaagccactttggaactcaaaaagaaaaatgaatcggagtcgccgaaaac